(19) United States
(12) Patent Application Publication
(10) Pub. No.: US 2017/0166896 A1
(43) Pub. Date: Jun. 15, 2017

(54) Treatment of Liver Cancer through Embolization Depot Delivery of BORIS Gene Silencing Agents

(71) Applicant: Regen Biopharma, Inc, La Mesa, CA (US)

(72) Inventors: David Koos, La Mesa, CA (US); Thomas ICHIM, San Diego, CA (US)

(21) Appl. No.: 15/250,877

(22) Filed: Aug. 29, 2016

Related U.S. Application Data
(60) Provisional application No. 62/211,605, filed on Aug. 28, 2015.

(51) Int. Cl. C12N 15/113; A61K 47/18; A61K 45/06; A61K 31/713

(52) U.S. Cl. CPC C12N 15/1136; A61K 31/713; A61K 47/18; A61K 45/06; C12N 2310/14; C12N 2320/31; C12N 2310/531; C12N 2320/32

(57) ABSTRACT

Methods of treatment of cancer are disclosed through administration of siRNA and shRNA sequences silencing BORIS gene and isoforms thereof. One embodiment of the invention discloses pharmaceutical compositions and kits for modifying the palliative procedure of transarterial chemoembolization so as to promote uptake of gene silencing inducing agents into the hepatic cancer microenvironment. By selectively administering under localized increased pressure, enhanced uptake of gene silencing agents is achieved, thus increasing targeting of tumor cells, particularly stem cells.

### CROSS-REFERENCE TO RELATED APPLICATIONS

This application is a non-provisional of and claims priority back to U.S. Provisional Application No. 62/211,605 filed Aug. 28, 2015, which is hereby incorporated by reference in its entirety.

### FIELD OF THE INVENTION

The present invention relates in general to the field of cancer gene silencing. Specifically, the invention relates to the field of localized gene silencing for cancer. Even more specifically, the invention relates to the field of initiating, augmenting and maintaining gene silencing in the tumor tissue.

### BACKGROUND

Localized killing of tumor cells is one means of not only eradicating tumors but also inducing immunity to tumors that is systemic. Immunological control of neoplasia is suggested by: A) Evidence of longer survival of patients with a variety of cancers who possess a high population of tumor infiltrating lymphocytes (1-3); B) The fact that immune suppressed patients develop cancer at a much higher frequency in comparison to non-immune suppressed individuals (4, 5); and C) In some very particular situations immunotherapy of cancer is clinically effective (6).

Transarterial chemoembolization (TACE), or otherwise defined as transcatheter chemoembolization, is a clinical procedure used primarily for treating primary and secondary liver cancer (7). TACE is usually employed when standard therapy has failed or is known to be ineffective. TACE combines the advantages of intra-arterial chemotherapy, with the fact that embolization of the portal artery induces a preferential “starvation” of the tumor while sparing non-malignant hepatic tissue. Specifically, it is established that intra-arterial delivery of chemotherapy to the liver results in a tenfold higher intratumoral concentration as compared to administration through the portal vein (8). This is due in part to the observation that both primary and secondary liver tumors derive their blood supply preferentially from the hepatic artery (9). Anecdotal evidence suggested that embolization caused by thrombosis of the catheter during delivery of intraarterial chemotherapy as beneficial for inducing an improved tumor response. This prompted investigators to use surgical ablation (10) or angiographic embolization (11-13) to induce localized necrosis. Unfortunately, this approach, in absence of chemotherapy caused little effect on long-term survival. Therefore the advantages of TACE is that both localized delivery of chemotherapy to the tumor occurs, while at the same time, the tumor blood flow is embolized, causing local tumor necrosis (14).

Cell death in general is known to release a variety of antigens. Globally speaking, apoptotic cell death is associated with anti-inflammatory and in some cases tolerogenesis, whereas necrotic cell death is perceived by the immune system as a “danger signal”, and is associated with immune activation (15-19). Specific examples of the anti-inflammatory aspects of apoptotic cell death include: the production of IL-10 by apoptotic monocytes (20); suppression of inflammatory cytokines by apoptotic bodies in vitro (21, 22), observations that administration of apoptotic but not necrotic cell bodies can actually endow macrophages with active immune suppressive properties (23); and clinically administered apoptotic blood cells have been demonstrated successful for treatment of inflammation associated with advanced heart failure in a recent Phase II trial (24). Conversely, cellular necrosis is associated with release of a variety of innate immune activation signals such as heat shock proteins (25-27), HMGB1 (28), mRNA with endogenous secondary structures (29), and even DNA complexed with endogenous factors such as natural antibodies (30, 31). Therefore the induction of cellular necrosis caused by TACE induces a release of tumor antigens, which is picked up by the immune system. The release of tumor antigens in such situations is reported in the literature (32), however taking advantage of this antigen release in the therapeutic context has not been accomplished to date.

Although the in the case of hepatocellular carcinoma, tumor itself (33-36), and host cells infiltrating the tumor are known to be immune suppressive (37), the microenvironment in which TACE induces cellular necrosis is also normally immune suppressive. It is known that intrahepatic administration of antigens results in systemic immune deviation towards weak cellular immunity (38). For example it was demonstrated that administration of donor cells into the hepatic circulation resulted in prolonged, donor specific, graft acceptance in various models of transplantation (39-43). The localized immune suppressive effects of the liver are known to the transplant clinician in that liver transplant recipients require a lower degree of immune suppression as compared to other organs. Additionally, in various rodent strain combinations hepatic grafts are spontaneously accepted, while cardiac or renal are rejected (44-46). At a cellular level this is explained by the presence of immature hepatic DC (47, 48), the tolerogenic potential of liver sinusoidal endothelial cells (49, 50), as well as natural killer T cells with a predisposition for releasing IL-4 (51, 52). Based on this, a release of tumor antigens within the hepatic microenvironment is postulated to cause a Th2, or immune regulatory shift, thereby not only failing to initiate protective immunity towards micrometastasis, but in some cases maybe even increasing the rate of tumor growth, through the phenomena of “tumor enhancement” described by Prehn (53).

Accordingly, there exists a need to “reprogram” the local immune environment in areas of tumor antigen release, so as to stimulate a productive immunity, which will cause systemic immunological control of neoplasia.

RNA interference (RNAi) is a process by which a double-stranded RNA (dsRNA) selectively inactivates homologous mRNA transcripts. The initial suggestion that dsRNA may possess such a gene silencing effect came from work in Petunias in which overexpression of the gene responsible for purple pigmentation actually caused the flower to lose their endogenous color (54). This phenomenon was termed co-suppression since both the inserted gene transcript and the endogenous transcript were suppressed. In 1998, Fire et al injected C. elegans with RNA in sense, antisense and the combination of both in order to suppress expression of several functional genes. Surprisingly, injection of the combined sense and antisense RNA led to more potent suppression of gene expression than sense or antisense used individually. Inhibition of gene expression was so potent that approximately 1-3 molecules of duplexed RNA per cell were effective at knocking down gene expression. Interestingly, suppression of gene expression would migrate from cell to cell and would even be passed from one generation of cells to another. This seminal paper was the first to describe RNAi (55). One problem present at the initial description of RNAi, and subsequent papers following, was that in order to induce RNAi, long pieces 200-800 base pairs, of dsRNA had to be used. This is impractical for therapeutic uses due to the sensitivity of long RNA to cleavage by RNAses found in the plasma and intracellularly. In addition, long pieces of dsRNA induce a panic response in eukaryotic cells, part of which includes nonspecific inhibition of gene transcription but production of interferon-α (56). In 2001, it was demonstrated that after a long dsRNA duplex enters the cytoplasm, a ribonuclease III type enzymatic activity cleaves the duplex into smaller, 21-23 base-pairs which are active in blocking endogenous gene expression. These small pieces of RNA, termed small interfering RNA (siRNA) are capable of blocking gene expression in mammalian cells without triggering the nonspecific panic response (57). Several studies published this year have used exogenously synthesized siRNA to block expression of disease associated genes in vitro. Novina et al demonstrated inhibition of HIV entry and replication using siRNA specific for CD4 and gag, respectively (58). Suppression of human papilloma virus gene expression in tissue biopsies from women with cervical carcinoma was reported using siRNA specific for the E6 and E7 genes (59). The first report of siRNA used in mammalian models is from McCaffrey et al who suppressed expression of luciferase in mice by administration of siRNA using a hydrodynamic transfection method (60). A subsequent study using HeLa cells xenografted on nude mice compared efficacy of gene suppression between AO and siRNA. Consistent with in vitro evidence, in vivo siRNA administration resulted in a more potent and longer lasting suppression of gene expression than obtained with AO (61). Silencing gene expression through siRNA is superior to conventional gene or antibody blocking approaches due to the following: 1) Blocking efficacy is potent (61); 2) Targeting gene expression is specific to 1 nucleotide mismatch (62); 3) Inhibitory effects can be passed for multiple generations to daughter cells (63); 4) In vitro transfection efficacy is higher and can be expressed in a stable manner (64); 5) In vivo use is more practical and safer due to lower concentrations needed and lack of neutralizing antibody production; 6) Tissue or cell specific gene targeting is possible using specific promoter vector (65, 66) or specific antibody conjugated liposomes; 7) Simultaneously targeting multiple genes or multiple exons silencing is possible for increasing efficacy (67).

One of the major limitations of RNA interference is that it requires systemic delivery which in many cases is difficult. In the current invention we leverage the localized “depot” approach of the TACE procedure to locally delivery siRNA to BORIS, thus causing tumor death. In contrast to chemotherapeutic agents that do not kill tumor stem cells, silencing of BORIS has previously been utilized to selectively kill tumor stem cells.

### DESCRIPTION OF THE INVENTION

In general, disclosed are methods and compositions useful for gene silencing, with the purpose of killing cancer stem cells or inducing differentiation, by localizing a depot of gene silencing nucleic acids such as siRNA with one strand possessing the sequence GGAAAUACCA CGAUGCAAAT (SEQ ID NO: 1) or in another embodiment one strand possessing the sequence

(SEQ ID NO: 2)
GGCAAGUAAA UUGAAGCGCT.

The term “a cell” as used herein includes a plurality of cells and refers to all types of cells including hematopoietic and cancer cells. Administering a compound to a cell includes in vivo, ex vivo and in vitro treatment.

The term “stem cell” as used herein refers to a cell that has the ability for self-renewal. Non-cancerous stem cells have the ability to differentiate where they can give rise to specialized cells.

The term “effective amount” as used herein means a quantity sufficient to, when administered to an animal, effect beneficial or desired results, including clinical results, and as such, an “effective amount” depends upon the context in which it is being applied. For example, in the context of inhibiting self-renewal of stem cells, it is the amount of the NR2F6 inhibitor sufficient to achieve such an inhibition as compared to the response obtained without administration of the NR2F6 inhibitor.

The term “oligonucleotide” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “animal” as used herein includes all members of the animal kingdom, preferably mammal. The term “mammal” as used herein is meant to encompass, without limitation, humans, domestic animals such as dogs, cats, horses, cattle, swine, sheep, goats, and the like, as well as wild animals. In an embodiment, the mammal is human.

The term “interfering RNA” or “RNAi” or “interfering RNA sequence” refers to double-stranded RNA (i.e., duplex RNA) that targets (i.e., silences, reduces, or inhibits) expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial or complete identity to the target gene. The sequence of the interfering RNA can correspond to the full length target gene, or a subsequence thereof. Interfering RNA includes small-interfering RNA” or “siRNA,” i.e., interfering RNA of about 15-60, 15-50, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, preferably about 20-24 or about 21-22 or 21-23 nucleotides in length, and the double stranded siRNA is about 15-60, 15-50, 15-50, 15-40, 15-30, 15-25 or 19-25 preferably about 20-24 or about 21-22 or 21-23 base pairs in length). siRNA duplexes may comprise 3′ overhangs of about 1 to about 4 nucleotides, preferably of about 2 to about 3 nucleotides and 5′ phosphate termini. The siRNA can be chemically synthesized or maybe encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99: 9942-7 (2002); Calegari et al., PNAS USA 99: 14236 (2002); Byrom et al., Ambion TechNotes 10(1): 4-6 (2003); Kawasaki et al., Nucleic Acids Res. 31: 981-7 (2003); Knight and Bass, Science 293: 2269-71 (2001); and Robertson et al., J. Biol. Chem. 243: 82 (1968)). Preferably, dsRNA are at least 50 nucleotides to about 100, 200, 300, 400 or 500 nucleotides in length. A dsRNA may be as long as 1000, 1500, 2000, 5000 nucleotides in length, or longer. The dsRNA can encode for an entire gene transcript or a partial gene transcript.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

The term “antisense nucleic acid” as used herein means a nucleotide sequence that is complementary to its target e.g. a NR2F6 transcription product. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

As used in this context, to “treat” means to ameliorate at least one symptom of the disorder. In some embodiments, a treatment can result in a reduction in tumor size or number, or a reduction in tumor growth or growth rate.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, e.g., affecting the nervous system, lung, breast, thyroid, lymphoid, gastrointestinal, and genito-urinary tract, as well as adenocarcinomas, which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

In the first aspect a method of treating cancer is disclosed, comprising the localized administration of an iodinated oil mixture, with an gene silencing agent capable of silencing expression of BORIS, or NR2F2 or NR2F6 together with an embolizing agent to a patient in need of therapy. The iodinated oil mixture could be the commonly used lipiodol solution, or novel derivatives thereof such as described in U.S. Pat. No. 6,690,962. The embolizing agent could be gelatin particles, or cyanoacrylate mixtures as described in U.S. Pat. No. 6,476,069. Additionally the use of other agents that induce either tumor cell necrosis or apoptosis, such as chemotherapeutic, radiotherapeutic, or agents that synergize with the aforementioned therapies may also be used to enhance localized cell death and antigen release. One skilled in the art would be familiar with Ohmoto et al who demonstrated utility of electromagnetic ablation together with TACE as a means of synergistically achieving tumor necrosis (80). Furthermore, prior to the embolization, agents may be administered either locally or systemically to enhance the expression of tumor antigens, said agents could include sodium phenylbutyrate, trinchostatin A, or 5-azacytidine. The administration of the mixture could be sequentially, concurrently, or in cycles. One type of administration would be through performing the transcatheter embolization procedure in a patient with primary hepatic cancer.

Another aspect of the invention is the addition of immune stimuli to the TACE procedure when it is being performed in the extra-hepatic context, for example in lung metastasis as described by Shitaba et al (81).

Another aspect of the invention involves administration of an agent capable of reducing levels of complement inhibitors on tumor cells, such as sodium phenylbutyrate (82), prior to and/or subsequent to administration of either conventional TACE or TACE together with a local immune stimulant.

Another aspect of the invention discloses compositions of matter suitable for use in stimulation of localized immune response. Such compositions involve a stable depot of immune stimulators such as TLR agonists, which program the immunological microenvironment to present tumor antigens in an immunostimulatory fashion in order to allow for induction of systemic immunity.

The foregoing has overviewed in a rather broad fashion the features and specific advantages of the present invention in order that the detailed description of the invention that follows may be better understood. Further specifics and methods of practicing the invention will be described afterwards, which comprise the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent embodiments or manifestations do not depart from the spirit and scope of the invention as set forth in the appended claims. The novel features which are believed to be characteristic of the invention, both as to its organization and method of operation, together with further objects and advantages will be better understood from the following description when considered in connection with the accompanying examples and the current state-of-the-art. It is to be understood, however, that each of the figures is provided for the purpose of illustration and description only and is not intended as a definition of the limits of the present invention.

Without intending to be limited by theory, the invention disclosed teaches methods of utilizing the immune response of a cancer patient in a therapeutic manner to control tumor recurrence and/or metastasis subsequent to a procedure during which tumor antigens are released.

Numerous procedures are clinically used that are associated with release of tumor antigens. Especially attractive procedures to which this invention is tailored are procedures associated with induction of tumor cell necrosis in a localized microenvironment. Specifically therapies such as transcatheter chemoembolization (TACE) conformal radiotherapy, percutaneous ethanol administration, embolization therapy, localized hyperthermia, and electromagnetic ablation therapy.

One specific embodiment of the invention involves modification of the TACE procedure in order to induce a systemic anti-tumor immunological effect. Specifically, patients are selected to meet the criteria for TACE. Said criteria includes: a) Adequate hepatic function; b) Patient portal vein circulation (confirmed during the venous phase of celiac or superior mesenteric angiogram); and c) Adequate renal function. Generally, only patients without cirrhosis or in Child group A or B disease are considered, however depending on experience of the practicing physician other groups may be included in the procedure as discussed by Shah et al (83). The TACE procedure may be performed either using a selective or superselective means. Patients selected to undergo the procedure receive 10 mg of phytonadione intravenously prior to the procedure (the intravenous injection should be administered slowly). Femoral catheterization and positioning of the catheter is performed. Premedication is with Lorazepam (Wyeth Laboratories, UK) 0.25 mg/kg orally 1 hour before the procedure to counter anxiety. An intra-arterial injection of 30-40 mg of 1% lidocaine is used for analgesia.

The following ingredients are made into an emulsion by repeatedly emptying and filling a syringe over 10 minutes: 10 mL of Lipiodol Ultrafluid (Mallinckrodt Medical, UK), 5 mL Omnipaque 300 (Amersham Health, UK; water-soluble contrast aids in emulsifying the mixture), 50 mg doxorubicin and clinical grade Poly (IC) stabilized with carboxymethylcellulose at a concentration between 0.025 mg/m² to 12 mg/m², preferably at a concentration of 0.2 mg/m². Intraarterial injection is administered under direct visualization to prevent reflux into gastroduodenal or splenic vessels. Embolization is performed with Ultra Ivalon 250-400 μm (Laboratories Nycomed SA). Intravenous cefuroxime (750 mg) and metronidazole (500 mg) are administered 3 times per day for 5 days. These antibiotics are given as prophylaxis against septicemia and liver abscess formation. Subsequent to administration patients are admitted to a high-dependency ward and should be mobilized after 6 hours of bedrest. Postoperative analgesia is administered if and when required by the patient. Patients also receive ranitidine (an H2 antagonist) intravenously 3 times per day until they begin eating. Patients are discharged home after 5 days or when their systemic symptoms begin resolving.

In order to monitor success of the procedure nonenhanced and enhanced CT examinations are performed 10-14 days following embolization. Furthermore, alpha-fetoprotein levels are evaluated at the 6-week outpatient review. If the TACE procedure is successful (>50% lipiodol uptake in necrotic tumor demonstrated on the postprocedural CT scan), the embolization is repeated in 6-8 weeks. Immunological monitoring is performed by assessing levels of interferon alpha production using ELISA during the 12, 24, and 72 hour time periods. Additionally, DTH, cellular and antibody responses are measured using pre-defined antigens representative of the tumor type.

A variety of chemotherapeutic agents can be used in practicing the invention. Specifically, chemotherapeutic agents which induce upregulation of costimulatory molecules are preferred. One example of such an agent is melphalan, which induces expression of CD80 on both tumor cells (84), as well as non-tumor B cells (85). In addition, a wide variety of chemotherapeutic agents are known in the art. These include: alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′, 2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; and capecitabine.

Tumors are usually associated with macrophage infiltration, this is correlated with tumor stage and is believed to contribute to tumor progression by stimulation of angiogenesis (107-109). Cytokines such as M-CSF (107) and VEGF (110) produced by tumor infiltrating macrophages are essential for tumor progression to malignancy. In fact, tumors implanted into M-CSF deficient op/op mice (that lack macrophages) do not metastasize or become vascularized (111). Tumor-associated macrophages possess an activated phenotype and release various inflammatory mediators such as cyclo-oxygenase metabolites (112, 113), TNF- [symbol] (114), and IL-6 (115) which lead to increased levels of oxidative stress produced by host immune cells. In addition, tumor associated macrophages themselves produce large amounts of free radicals such as NO, OH, and H₂O₂ (116-118). The high levels of macrophage activation in cancer patients is illustrated by high serum levels of neopterin, a tryptophan metabolite that is associated with poor prognosis (119). In addition to oxidative stress elaborated by tumor associated macrophages, the presence of the tumor itself causes systemic changes associated with chronic inflammation. Erythrocyte sedimentation ration, C-reactive protein and IL-6 are markers of inflammatory stress used to designate progression of pathological immune diseases such as arthritis (120, 121). Interestingly advanced cancer patients possess all of these inflammatory markers (122-126). Another marker of chronic inflammation is decreased albumin synthesis by the liver, this is also seen in cancer patients and is believed to contribute, at least in part, to cachexia (127, 128). In addition, the inflammatory marker fibrinogen D-dimers is also higher in cancer patients as opposed to controls (129-131). Schmielau et al reported that in patients with a variety of cancers, activated neutrophils are circulating in large numbers (101). These neutrophils secrete reactive oxygen radicals such as hydrogen peroxide, which trigger suppression of TCR-ζ and IFN-γ production. This was demonstrated by co-incubation of the neutrophils from cancer patients with lymphocytes from healthy volunteer. A profound suppression of TCR-ζ expression was seen. Evidence for the critical role of hydrogen peroxide was shown by the fact that addition of catalase suppressed TCR-ζ downregulation. A simple method of assessing the number of circulating activated neutrophils was described in the same paper. This method involves collecting peripheral blood from patients, spinning the blood on a density gradient such as Ficoll, and collecting the lymphocyte fraction. While in healthy volunteers the lymphocyte fraction contained primarily lymphocytes, in cancer patients the lymphocyte fraction contained both lymphocytes and a large number of neutrophils. The reason why these neutrophils are present in the lymphocyte fraction is because activation alters their density so that they co-purify differently on the gradient. A potential indication of the importance of activated neutrophils to cancer progression is provided by Tabuchi et al who show that removal of granulocytes from the peripheral blood of cancer patients resulted in reduced tumor size, unfortunately, the study was performed in only 2 patients (132). As a mechanism to compensate for immune over-activation, mediators of inflammation have immune suppressive properties. This is best illustrated in the immune suppression seen following immune hyperactivation such as in septic shock. Following the primary septicemia, patients are systemically immune compromised due to circulating immune suppressive factors that are released in response to the inflammatory stress. This suppression is termed compensatory anti-inflammatory response syndrome (CARS) and is associated with many opportunistic infections and deactivation (133). The clinical importance of CARS immune suppression is seen in that sepsis survivors show normal T-cell proliferation and IL-2 release, whereas those that succumb possess suppressed T cell responses (134). Interestingly immune suppressive mediators associated with CARS such as PGE2, TGF-β, and IL-10 are also associated with cancer-induced immune suppression (135). The role of oxidative stress in sepsis-induced immune suppression was recently demonstrated in experiments where administration of antioxidants (ascorbic acid or n-acetylcysteine) to animals undergoing experimental sepsis blocked immune suppression (136). Another example of the potential for antioxidants to stimulate immune response in an inflammatory condition is in patients with Duke's C and D colorectal cancer who were administered of a daily dose of 750 mg of vitamin E for 2 weeks. This resulted in restoration of IFN-γ and IL-2 production (137). The problem of uncontrolled inflammation is seen in sepsis. Although as a monotherapy n-acetylcysteine has little clinical effect, therapeutic administration of n-acetylcysteine results in suppression of the constitutively activated neutrophils seen in these patients (138). Administration of n-acetylcysteine to smokers results in suppression of markers of oxidative stress (139). Furthermore, oral n-acetylcysteine administration blocks angiogenesis and suppresses growth of Kaposi Sarcoma (140). Accordingly, a method of preparing the host for the TACE procedure includes administration of n-acetylcysteine at a concentration sufficient to decrease the tumor associated suppression of T cell activity. Such a concentration ranges between 1-10 grams per day, preferably 4-6 grams administered intravenously for a period of type sufficient to normalize production of IFN-γ from PBMC of cancer patients upon ex vivo stimulation. One skilled in the art will understand that n-acetylcysteine is just one example of a compound suitable for reversion of oxidative-stress associated immune suppression. Numerous other compounds may be used, for example ascorbic acid (141-143), co-enzyme Q10 in combination with vitamin E and alpha-lipoic acid (144), genistein (145) or resveratrol (146).

CD4⁺ CD25⁺ T regulatory cells (Treg) are considered to be a “mirror-immune system” capable of recognizing a similar repertoire of antigens as conventional T cells, with the exception that instead of inducing immune activation, they suppress it (147). Treg cells are generated in the thymus by positive selection to self antigens, whereas conventional T cells are deleted intrathymically upon recognition of self antigens (148). Specifically, the Hassall's corpuscle of the thymus was demonstrated to be the site of self-antigen reactive Treg generation (149). Additionally, Treg cells are generated in the periphery in response to self antigens being presented on tolerogenic or immature dendritic cells in the basal state or in situations of tolerance induction (150). Treg cells are capable of suppressing T helper (151), T cytotoxic (152), T memory (153), and NKT cell function (154), as well as ability of DC to mature (155) through a variety of mechanisms including surface bound TGF- [symbol] (156), granzyme B secretion (157), and IL-10 release (158).

One specific embodiment of the invention is administration of siRNA specific to an immune suppressive factor directly into tumors using a catheter-based delivery approach. Co-administration of the siRNA-lipiodol mixture with embolization, and/or chemotherapy is envisioned within the scope of the invention. A specific application of the invention is generation of siRNA targeting the immune suppressive enzyme indoleamine 2,3-dioxygenase (IDO) (186), and administering said siRNA via hepatic artery embolization into a patient with liver cancer. Targeting of IDO mRNA transcript is particularly advantageous since in addition to endogenous tumor expression of IDO, host cells upregulate expression of this enzyme in response to immune activation as a negative feedback loop (187). Accordingly the silencing of IDO in a cancer patient concurrently with systemic or local immune stimulation can be utilized for synergistic immune enhancement. Numerous other cytokines, transcription factors, and membrane-bound immune suppressive factors can be silenced within the context of the disclosed invention in order to augment immune activation subsequent to induction of localized cell death. Examples of relevant immune suppressive factors associated with neoplasia include: IL-10 (188), TGF- [symbol] (189), Fas ligand (190), VEGF (191), IL-18 binding protein (192), MUC-1 (193), decoy receptor 3 (194), sigma(1) receptors (195), heavy chain ferritin (196), angiotensin II type I receptor (197), STATE (198), or protectin/CD59 (199). In one preferred embodiment of the invention, silencing of genes associated with cancer stem cells is performed, said genes are selected from a group comprising of: a) BORIS; b) NR2F6; c) NR2F2; d) telomerase and e) NOTCH.

In one embodiment, nucleic acids provided herein can include both unmodified siRNAs and modified siRNAs as known in the art. For example, in some embodiments, siRNA derivatives can include siRNA having two complementary strands of nucleic acid, such that the two strands are crosslinked. For a specific example, a 3′ OH terminus of one of the strands can be modified, or the two strands can be crosslinked and modified at the 3′ OH terminus. The siRNA derivative can contain a single crosslink (one example of a useful crosslink is a psoralen crosslink). In some embodiments, the siRNA derivative has at its 3′ terminus a biotin molecule (for example, a photocleavable molecule such as biotin), a peptide (as an example an HIV Tat peptide), a nanoparticle, a peptidomimetic, organic compounds, or dendrimer. Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acids described within the practice of the current invention can include nucleic acids that are unconjugated or can be conjugated to another moiety, such as a nanoparticle, to enhance a desired property of the pharmaceutical composition. Properties useful in the development of a therapeutic agent include: a) absorption; b) efficacy; c) bioavailability; and d) half life in blood or in vivo. RNAi is believed to progress via at least one single stranded RNA intermediate, the skilled artisan will appreciate that single stranded-siRNAs (e.g., the antisense strand of a ds-siRNA) can also be designed as described herein and utilized according to the claimed methodologies.

In one embodiment the pharmaceutical composition comprises a nucleic acid-lipid particle that contains an siRNA oligonucleotide that induces RNA interference against NR2F6. In some aspects the lipid portion of the particle comprises a cationic lipid and a non-cationic lipid. In some aspects the nucleic acid-lipid particle further comprises a conjugated lipid that prevents aggregation of the particles and/or a sterol (e.g., cholesterol).

For practice of the invention, methods for expressing siRNA duplexes within cells from recombinant DNA constructs to allow longer-term target gene suppression in cells are known in the art, including mammalian Pol III promoter systems (e.g., H1 or U6/snRNA promoter systems) capable of expressing functional double-stranded siRNAs. Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression. Constructs containing siRNA sequence(s) under the control of a T7 promoter also make functional siRNAs when co-transfected into the cells with a vector expressing T7 RNA polymerase. A single construct may contain multiple sequences coding for siRNAs, such as multiple regions of the NR2F6 gene, such as a nucleic acid encoding the NR2F6 mRNA, and can be driven, for example, by separate Pol III promoter sites. In some situations it will be preferable to induce expression of the hairpin siRNA or shRNAs in a tissue specific manner in order to activate the shRNA transcription that would subsequently silence NR2F6 expression. Tissue specificity may be obtained by the use of regulatory sequences of DNA that are activated only in the desired tissue. Regulatory sequences include promoters, enhancers and other expression control elements such as polyadenylation signals. Regulatory sequences include those which direct constitutive expression of a nucleotide sequence in many types of host cell and those which direct expression of the nucleotide sequence only in certain host cells. Tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters as follows may be used to target gene expression in other tissues. Examples of more tissue specific promoters include in (a) to target the pancreas promoters for the following may be used: insulin, elastin, amylase, pdr-I, pdx-I, glucokinase; (b) to target the liver promoters for the following may be used: albumin PEPCK, HBV enhancer, a fetoprotein, apolipoprotein C, .alpha.-I antitrypsin, vitellogenin, NF-AB, Transthyretin; (c) to target the skeletal muscle promoters for the following may be used: myosin H chain, muscle creatine kinase, dystrophin, calpain p94, skeletal .alpha.-actin, fast troponin 1; (d) to target the skin promoters for the following may be used: keratin K6, keratin KI; (e) lung: CFTR, human cytokeratin IS (K 18), pulmonary surfactant proteins A, B and C, CC-10, Pi; (0 smooth muscle: sm22 .alpha., SM-.alpha.-actin; (g) to target the endothelium promoters for the following may be used: endothelin-I, E-selectin, von Willebrand factor, TIE, KDR/flk-I; (h) to target melanocytes the tyrosinase promoter may be used; (i) to target the mammary gland promoters for the following may be used: MMTV, and whey acidic protein (WAP).

Yet another embodiment of the invention consists of a pharmaceutical composition comprising an oligonucleotide that induces RNA interference against NR2F6 combined with a delivery agent such as a liposome. For more targeted delivery immunoliposomes, or liposomes containing an agent inducing selective binding to neoplastic cells may be used.

The present invention further provides pharmaceutical compositions comprising the nucleic acid-lipid particles described herein and a pharmaceutically acceptable carrier.

Another embodiment of the invention consists of a pharmaceutical composition comprising an oligonucleotide that induces RNA interference against NR2F6 combined with an additional chemotherapeutic agent.

Yet another embodiment of the invention consists of a pharmaceutical composition comprising an oligonucleotide that induces RNA interference against NR2F6 combined with an additional agent used to induce differentiation

One embodiment of the invention is a short-interfering ribonucleic acid (siRNA) molecule effective at silencing NR2F6 expression that has been cloned in to an appropriate expression vector giving rise to an shRNA vector.

In certain embodiment shRNA oligonucleotides are cloned in to an appropriate mammalian expression vectors, examples of appropriate vectors include but are not limited to lentiviral, retroviral or adenoviral vector.

In this embodiment, the invention consists of a viral vector, comprising the inhibitory RNA molecule described above. The viral vector preferably is a lentivirus. In one aspect the viral vector is capable of infecting cancer cells. Another embodiment is a lentivirus vector that is an integrating vector. The viral vector preferably is capable of transducing cancer cells. The viral vector is preferably packaged in a coat protein the specifically binds to cancer cells. The viral vector preferably is capable of expressing an RNA that inhibits NR2F6 expression. Another embodiment of the invention is one in which the viral vector is preferably produced by a vector transfer cassette and a separate helper plasmid. In certain embodiment the shRNA oligonucleotides is combined with a pharmaceutically acceptable vehicle a pharmaceutical composition. One embodiment is a pharmaceutical composition comprising an inhibitory oligonucleotide that is a double stranded RNA molecule.

One aspect of the invention is a microRNA or family of microRNAs are administered that substantially inhibit expression of NR2F6

siRNA may be created using a variety of chemical synthesis methods known to one skilled in the art. Such methods can include addition of phosphorothioate internucleotide linkages, 2′-O-methyl ribonucleotides, 2′-deoxy-2′-fluoro ribonucleotides, “universal base” nucleotides, 5-C-methyl nucleotides, and inverted deoxyabasic residue incorporation. Chemical modifications of the siRNA constructs can also be used to improve the stability of the interaction with the target RNA sequence and to improve nuclease resistance.

In one embodiment, the invention features a chemically modified short interfering siRNA wherein the chemical modification comprises a conjugate covalently attached to the siRNA molecule. In another embodiment, the conjugate is covalently attached to the siRNA molecule via a linker, said linker being degradable within the host or host cells. The conjugate molecule is attached at the 3′-end of either the sense strand, antisense strand, or both strands of the siRNA. The conjugate molecule is attached at the 5′-end of either the sense strand, antisense strand, or both strands of the siRNA. Alternatively the conjugate molecule is attached both the 3′-end and 5′-end of either the sense strand, antisense strand, or both strands of the siRNA, or any combination thereof. In one embodiment, a conjugate molecule of the invention comprises a molecule that facilitates delivery of a siRNA molecule into the tumor cell or host cell surrounding the tumor. In another embodiment, the conjugate molecule attached to the siRNA is a poly ethylene glycol, human serum albumin, or a ligand for a cellular receptor found either on the cancer cell or the proximal host cell that can mediate cellular uptake.

### REFERENCES

All references cited throughout the disclosure, and all references cited therein are hereby expressly incorporated by reference in their entirety.
1. Ryschich, E., Notzel, T., Hinz, U., Autschbach, F., Ferguson, J., Simon, I., Weitz, J., Frohlich, B., Klar, E., Buchler, M. W., et al. 2005. Control of T-cell-mediated immune response by HLA class I in human pancreatic carcinoma. Clin Cancer Res 11:498-504.
2. Raspollini, M. R., Castiglione, F., Rossi Degl'innocenti, D., Amunni, G., Villanucci, A., Garbini, F., Baroni, G., and Taddei, G. L. 2005. Tumour-infiltrating gamma/delta T-lymphocytes are correlated with a brief disease-free interval in advanced ovarian serous carcinoma. Ann Oncol 16:590-596.
3. Chiba, T., Ohtani, H., Mizoi, T., Naito, Y., Sato, E., Nagura, H., Ohuchi, A., Ohuchi, K., Shiiba, K., Kurokawa, Y., et al. 2004. Intraepithelial CD8+ T-cell-count becomes a prognostic factor after a longer follow-up period in human colorectal carcinoma: possible association with suppression of micrometastasis. Br J Cancer 91:1711-1717.
4. Astigiano, S., Morandi, B., Costa, R., Mastracci, L., D'Agostino, A., Ratto, G. B., Melioli, G., and Frumento, G. 2005. Eosinophil granulocytes account for indoleamine 2,3-dioxygenase-mediated immune escape in human non-small cell lung cancer. Neoplasia 7:390-396.
5. Whiteside, T. L. 2004. Down-regulation of zeta-chain expression in T cells: a biomarker of prognosis in cancer? Cancer Immunol Immunother 53:865-878.
6. Rosenberg, S. A., and Dudley, M. E. 2004. Cancer regression in patients with metastatic melanoma after the transfer of autologous antitumor lymphocytes. Proc Natl Acad Sci USA 101 Suppl 2:14639-14645.
7. Ramsey, D. E., Kernagis, L. Y., Soulen, M. C., and Geschwind, J. F. 2002. Chemoembolization of hepatocellular carcinoma. J Vasc Interv Radiol 13:S211-221.
8. Sigurdson, E. R., Ridge, J. A., Kemeny, N., and Daly, J. M. 1987. Tumor and liver drug uptake following hepatic artery and portal vein infusion. J Clin Oncol 5:1836-1840.
9. Breedis, C., and Young, G. 1954. The blood supply of neoplasms in the liver. Am J Pathol 30:969-977.
10. McDermott, W. V., Jr., Paris, A. L., Clouse, M. E., and Meissner, W. A. 1978. Dearterialization of the liver for metastatic cancer. Clinical, angiographic and pathologic observations. Ann Surg 187:38-46.
11. Clouse, M. E., Lee, R. G., Duszlak, E. J., Lokich, J. J., and Alday, M. T. 1983. Hepatic artery embolization for metastatic endocrine-secreting tumors of the pancreas. Report of two cases. Gastroenterology 85:1183-1186.
12. Nakao, N., Miura, K., Takahashi, H., Ohnishi, M., Miura, T., Okamoto, E., and Ishikawa, Y. 1986. Hepatocellular carcinoma: combined hepatic, arterial, and portal venous embolization. Radiology 161:303-307.
13. Hwang, T. L., Chen, M. F., Lee, T. Y., Chen, T. J., Lin, D. Y., and Liaw, Y. F. 1987. Resection of hepatocellular carcinoma after transcatheter arterial embolization. Reevaluation of the advantages and disadvantages of preoperative embolization. Arch Surg 122:756-759.
14. Stuart, K. 2003. Chemoembolization in the management of liver tumors. Oncologist 8:425-437.
15. Pulendran, B. 2004. Immune activation: death, danger and dendritic cells. Curr Biol 14:R30-32.
16. Rock, K. L., Hearn, A., Chen, C. J., and Shi, Y. 2005. Natural endogenous adjuvants. Springer Semin Immunopathol 26:231-246.
17. McBride, W. H., Chiang, C. S., Olson, J. L., Wang, C. C., Hong, J. H., Pajonk, F., Dougherty, G. J., Iwamoto, K. S., Pervan, M., and Liao, Y. P. 2004. A sense of danger from radiation. Radiat Res 162:1-19.
18. Friedman, E. J. 2002. Immune modulation by ionizing radiation and its implications for cancer immunotherapy. Curr Pharm Des 8:1765-1780.
19. Sauter, B., Albert, M. L., Francisco, L., Larsson, M., Somersan, S., and Bhardwaj, N. 2000. Consequences of cell death: exposure to necrotic tumor cells, but not primary tissue cells or apoptotic cells, induces the maturation of immunostimulatory dendritic cells. J Exp Med 191:423-434.
20. Bzowska, M., Guzik, K., Barczyk, K., Ernst, M., Flad, H. D., and Pryjma, J. 2002. Increased IL-10 production during spontaneous apoptosis of monocytes. Eur J Immunol 32:2011-2020.
21. Cvetanovic, M., and Ucker, D. S. 2004. Innate immune discrimination of apoptotic cells: repression of proinflammatory macrophage transcription is coupled directly to specific recognition. J Immunol 172:880-889.
22. Hoffmann, P. R., Kench, J. A., Vondracek, A., Kruk, E., Daleke, D. L., Jordan, M., Marrack, P., Henson, P. M., and Fadok, V. A. 2005. Interaction between phosphatidylserine and the phosphatidylserine receptor inhibits immune responses in vivo. J Immunol 174:1393-1404.
23. Reiter, I., Krammer, B., and Schwamberger, G. 1999. Cutting edge: differential effect of apoptotic versus necrotic tumor cells on macrophage antitumor activities. J Immunol 163:1730-1732.
24. Torre-Amione, G., Sestier, F., Radovancevic, B., and Young, J. 2004. Effects of a novel immune modulation therapy in patients with advanced chronic heart failure: results of a randomized, controlled, phase II trial. J Am Coll Cardiol 44:1181-1186.
25. Basu, S., Binder, R. J., Suto, R., Anderson, K. M., and Srivastava, P. K. 2000. Necrotic but not apoptotic cell death releases heat shock proteins, which deliver a partial maturation signal to dendritic cells and activate the NF-kappa B pathway. Int Immunol 12:1539-1546.
26. Quintana, F. J., and Cohen, I. R. 2005. Heat shock proteins as endogenous adjuvants in sterile and septic inflammation. J Immunol 175:2777-2782.
27. Tsan, M. F., and Gao, B. 2004. Endogenous ligands of Toll-like receptors. J Leukoc Biol 76:514-519.
28. Rovere-Querini, P., Capobianco, A., Scaffidi, P., Valentinis, B., Catalanotti, F., Giazzon, M., Dumitriu, I. E., Muller, S., Iannacone, M., Traversari, C., et al. 2004. HMGB1 is an endogenous immune adjuvant released by necrotic cells. EMBO Rep 5:825-830.
29. Kariko, K., Ni, H., Capodici, J., Lamphier, M., and Weissman, D. 2004. mRNA is an endogenous ligand for Toll-like receptor 3. J Biol Chem 279:12542-12550.
30. Barrat, F. J., Meeker, T., Gregorio, J., Chan, J. H., Uematsu, S., Akira, S., Chang, B., Duramad, O., and Coffman, R. L. 2005. Nucleic acids of mammalian origin can act as endogenous ligands for Toll-like receptors and may promote systemic lupus erythematosus. J Exp Med 202:1131-1139.
31. Christensen, S. R., Kashgarian, M., Alexopoulou, L., Flavell, R. A., Akira, S., and Shlomchik, M. J. 2005. Toll-like receptor 9 controls anti-DNA autoantibody production in murine lupus. J Exp Med 202:321-331.
32. Wu, F., Wang, Z. B., Lu, P., Xu, Z. L., Chen, W. Z., Zhu, H., and Jin, C. B. 2004. Activated anti-tumor immunity in cancer patients after high intensity focused ultrasound ablation. Ultrasound Med Biol 30:1217-1222.
33. Ormandy, L. A., Hillemann, T., Wedemeyer, H., Manns, M. P., Greten, T. F., and Korangy, F. 2005. Increased populations of regulatory T cells in peripheral blood of patients with hepatocellular carcinoma. Cancer Res 65:2457-2464.
34. Jessup, J. M., Samara, R., Battle, P., and Laguinge, L. M. 2004. Carcinoembryonic antigen promotes tumor cell survival in liver through an IL-10-dependent pathway. Clin Exp Metastasis 21:709-717.
35. Yuan, L., Kobayashi, M., Kuramitsu, Y., Li, Y., Matsushita, K., and Hosokawa, M. 1997. Restoration of macrophage tumoricidal activity by bleomycin correlates with the decreased production of transforming growth factor beta in rats bearing KDH-8 hepatoma cells. Cancer Immunol Immunother 45:71-76.
36. Sondak, V. K., Wagner, P. D., Shu, S., and Chang, A. E. 1991. Suppressive effects of visceral tumor on the generation of antitumor T cells for adoptive immunotherapy. Arch Surg 126:442-446.
37. Griffini, P., Smorenburg, S. M., Vogels, I. M., Tigchelaar, W., and Van Noorden, C. J. 1996. Kupffer cells and pit cells are not effective in the defense against experimentally induced colon carcinoma metastasis in rat liver. Clin Exp Metastasis 14:367-380.
38. Crispe, I. N., Dao, T., Klugewitz, K., Mehal, W. Z., and Metz, D. P. 2000. The liver as a site of T-cell apoptosis: graveyard, or killing field? Immunol Rev 174:47-62.
39. Kara, E., Gokhan, I., Dayangac, M., Ilkgul, O., Ertan, H., Tokat, Y., and Terzioglu, E. 2004. Effect of portal venous injection of donor spleen cells on skin allograft survival in rat. Indian J Med Res 119:110-114.
40. Yu, S., Nakafusa, Y., and Flye, M. W. 1994. Portal vein administration of donor cells promotes peripheral allospecific hyporesponsiveness and graft tolerance. Surgery 116:229-234; discussion 234-225.
41. Hamashima, T., Yoshimura, N., Matsui, S., Lee, C. J., and Oka, T. 1989. [Effects of portal venous administration with allogenic cells on renal allograft survival in the rat]. Nippon Geka Gakkai Zasshi 90:1752-1757.
42. Nakano, Y., Monden, M., Valdivia, L. A., Gotoh, M., Tono, T., and Mori, T. 1992. Permanent acceptance of liver allografts by intraportal injection of donor spleen cells in rats. Surgery 111:668-676.
43. Carr, R. I., Zhou, J., Ledingham, D., Maloney, C., McAlister, V., Samson, M., Bitter-Suermann, H., and Lee, T. D. 1996. Induction of transplantation tolerance by feeding or portal vein injection pretreatment of recipient with donor cells. Ann N Y Acad Sci 778:368-370.
44. Asakura, H., Takayashiki, T., Ku, G., and Flye, M. W. 2005. The persistence of regulatory cells developing after rat spontaneous liver acceptance. Surgery 138:329-334.
45. Reding, R., and Davies, H. F. 2004. Revisiting liver transplant immunology: from the concept of immune engagement to the dualistic pathway paradigm. Liver Transpl 10:1081-1086.
46. Delriviere, L., Havaux, X., Latinne, D., Bazin, H., Kamada, N., Nordlinger, B., and Gianello, P. 1997. Administration of exogenous interleukin-2 abrogates spontaneous rat liver allograft acceptance but does not affect long-term established graft survival. Transplantation 63:1698-1701.
47. den Dulk, M., and Bishop, G. A. 2003. Immune mechanisms contributing to spontaneous acceptance of liver transplants in rodents and their potential for clinical transplantation. Arch Immunol Ther Exp (Warsz) 51:29-44.
48. Steptoe, R. J., Fu, F., Li, W., Drakes, M. L., Lu, L., Demetris, A. J., Qian, S., McKenna, H. J., and Thomson, A. W. 1997. Augmentation of dendritic cells in murine organ donors by Flt3 ligand alters the balance between transplant tolerance and immunity. J Immunol 159:5483-5491.
49. Limmer, A., Ohl, J., Wingender, G., Berg, M., Jungerkes, F., Schumak, B., Djandji, D., Scholz, K., Klevenz, A., Hegenbarth, S., et al. 2005. Cross-presentation of oral antigens by liver sinusoidal endothelial cells leads to CD8 T cell tolerance. Eur J Immunol 35:2970-2981.
50. Onoe, T., Ohdan, H., Tokita, D., Shishida, M., Tanaka, Y., Hara, H., Zhou, W., Ishiyama, K., Mitsuta, H., Ide, K., et al. 2005. Liver sinusoidal endothelial cells tolerize T cells across MEW barriers in mice. J Immunol 175:139-146.
51. Crispe, I. N. 2003. Hepatic T cells and liver tolerance. Nat Rev Immunol 3:51-62.
52. Sharif, S., Arreaza, G. A., Zucker, P., Mi, Q. S., Sondhi, J., Naidenko, O. V., Kronenberg, M., Koezuka, Y., Delovitch, T. L., Gombert, J. M., et al. 2001. Activation of natural killer T cells by alpha-galactosylceramide treatment prevents the onset and recurrence of autoimmune Type 1 diabetes. Nat Med 7:1057-1062.
53. Prehn, R. T. 1972. Proceedings: Immune involvement in oncogenesis. Proc Natl Cancer Conf 7:401-404.
54. Jorgensen, R. A., Cluster, P. D., English, J., Que, Q., and Napoli, C. A. 1996. Chalcone synthase cosuppression phenotypes in petunia flowers: comparison of sense vs. antisense constructs and single-copy vs. complex T-DNA sequences. Plant Mol Biol 31:957-973.
55. Fire, A., Xu, S., Montgomery, M. K., Kostas, S. A., Driver, S. E., and Mello, C. C. 1998. Potent and specific genetic interference by double-stranded RNA in Caenorhabditis elegans. Nature 391:806-811.
56. Proud, C. G. 1995. PKR: a new name and new roles. Trends Biochem Sci 20:241-246.
57. Elbashir, S. M., Harborth, J., Lendeckel, W., Yalcin, A., Weber, K., and Tuschl, T. 2001. Duplexes of 21-nucleotide RNAs mediate RNA interference in cultured mammalian cells. Nature 411:494-498.
58. Novina, C. D., Murray, M. F., Dykxhoorn, D. M., Beresford, P. J., Riess, J., Lee, S. K., Collman, R. G., Lieberman, J., Shankar, P., and Sharp, P. A. 2002. siRNA-directed inhibition of HIV-1 infection. Nat Med 8:681-686.
59. Jiang, M., and Milner, J. 2002. Selective silencing of viral gene expression in HPV-positive human cervical carcinoma cells treated with siRNA, a primer of RNA interference. Oncogene 21:6041-6048.
60. McCaffrey, A. P., Meuse, L., Pham, T. T., Conklin, D. S., Hannon, G. J., and Kay, M. A. 2002. RNA interference in adult mice. Nature 418:38-39.
61. Bertrand, J., Pottier, M., Vekris, A., Opolon, P., Maksimenko, A., and Malvy, C. 2002. Comparison of antisense oligonucleotides and siRNAs in cell culture and in vivo. Biochem Biophys Res Commun 296:1000.
62. Celotto, A. M., and Graveley, B. R. 2002. Exon-specific RNAi: a tool for dissecting the functional relevance of alternative splicing. Rna 8:718-724.
63. Grishok, A., Tabara, H., and Mello, C. C. 2000. Genetic requirements for inheritance of RNAi in C. elegans. Science 287:2494-2497.
64. Brummelkamp, T. R., Bernards, R., and Agami, R. 2002. A system for stable expression of short interfering RNAs in mammalian cells. Science 296:550-553.
65. Paul, C. P., Good, P. D., Winer, I., and Engelke, D. R. 2002. Effective expression of small interfering RNA in human cells. Nat Biotechnol 20:505-508.
66. Devroe, E., and Silver, P. A. 2002. Retrovirus-delivered siRNA. BMC Biotechnol 2:15.
67. Yang, D., Buchholz, F., Huang, Z., Goga, A., Chen, C. Y., Brodsky, F. M., and Bishop, J. M. 2002. Short RNA duplexes produced by hydrolysis with Escherichia coli RNase III mediate effective RNA interference in mammalian cells. Proc Natl Acad Sci USA 99:9942-9947.
68. Botti, C., Seregni, E., Ferrari, L., Martinetti, A., and Bombardieri, E. 1998. Immunosuppressive factors: role in cancer development and progression. Int J Biol Markers 13:51-69.
69. Gilboa, E. 1999. How tumors escape immune destruction and what we can do about it. Cancer Immunol Immunother 48:382-385.
70. Yang, L., and Carbone, D. P. 2004. Tumor-host immune interactions and dendritic cell dysfunction. Adv Cancer Res 92:13-27.
71. Takahashi, A., Kono, K., Ichihara, F., Sugai, H., Amemiya, H., Iizuka, H., Fujii, H., and Matsumoto, Y. 2003. Macrophages in tumor-draining lymph node with different characteristics induce T-cell apoptosis in patients with advanced stage-gastric cancer. Int J Cancer 104:393-399.
72. Sanchez-Fueyo, A. 2005. [Immunological tolerance and liver transplantation]. Gastroenterol Hepatol 28:250-256.
73. Parker, G. A., and Picut, C. A. 2005. Liver immunobiology. Toxicol Pathol 33:52-62.
74. Nikitina, E. Y., and Gabrilovich, D. I. 2001. Combination of gamma-irradiation and dendritic cell administration induces a potent antitumor response in tumor-bearing mice: approach to treatment of advanced stage cancer. Int J Cancer 94:825-833.
75. Teitz-Tennenbaum, S., Li, Q., Rynkiewicz, S., Ito, F., Davis, M. A., McGinn, C. J., and Chang, A. E. 2003. Radiotherapy potentiates the therapeutic efficacy of intratumoral dendritic cell administration. Cancer Res 63:8466-8475.
76. den Brok, M. H., Sutmuller, R. P., van der Voort, R., Bennink, E. J., Figdor, C. G., Ruers, T. J., and Adema, G. J. 2004. In situ tumor ablation creates an antigen source for the generation of antitumor immunity. Cancer Res 64:4024-4029.
77. Soanes, W. A., Ablin, R. J., and Gonder, M. J. 1970. Remission of metastatic lesions following cryosurgery in prostatic cancer: immunologic considerations. J Urol 104:154-159.
78. Sanchez-Ortiz, R. F., Tannir, N., Ahrar, K., and Wood, C. G. 2003. Spontaneous regression of pulmonary metastases from renal cell carcinoma after radio frequency ablation of primary tumor: an in situ tumor vaccine? J Urol 170:178-179.
79. Mercader, M., Bodner, B. K., Moser, M. T., Kwon, P. S., Park, E. S., Manecke, R. G., Ellis, T. M., Wojcik, E. M., Yang, D., Flanigan, R. C., et al. 2001. T cell infiltration of the prostate induced by androgen withdrawal in patients with prostate cancer. Proc Natl Acad Sci USA 98:14565-14570.
80. Ohmoto, K., Yoshioka, N., Tomiyama, Y., Shibata, N., Kawase, T., Yoshida, K., Kuboki, M., and Yamamoto, S. 2005. Carbon dioxide-enhanced sonographically guided radiofrequency ablation combined with transcatheter arterial chemoembolization for sonographically undetectable hepatocellular carcinoma. Hepatogastroenterology 52:1344-1346.
81. Shibata, T., Maetani, Y., Kubo, T., Nishida, N., and Itoh, K. 2005. Transcatheter Arterial Embolization for Tumor Seeding in the Chest Wall After Radiofrequency Ablation for Hepatocellular Carcinoma. Cardiovasc Intervent Radiol.
82. Andoh, A., Shimada, M., Araki, Y., Fujiyama, Y., and Bamba, T. 2002. Sodium butyrate enhances complement-mediated cell injury via down-regulation of decay-accelerating factor expression in colonic cancer cells. Cancer Immunol Immunother 50:663-672.
83. Shah, S. R., Riordan, S. M., Karani, J., and Williams, R. 1998. Tumour ablation and hepatic decompensation rates in multi-agent chemoembolization of hepatocellular carcinoma. Qjm 91:821-828.
84. Donepudi, M., Raychaudhuri, P., Bluestone, J. A., and Mokyr, M. B. 2001. Mechanism of melphalan-induced B7-1 gene expression in P815 tumor cells. J Immunol 166:6491-6499.
85. Donepudi, M., Jovasevic, V. M., Raychaudhuri, P., and Mokyr, M. B. 2003. Melphalan-induced up-regulation of B7-1 surface expression on normal splenic B cells. Cancer Immunol Immunother 52:162-170.
86. Ng, C. S., Novick, A. C., Tannenbaum, C. S., Bukowski, R. M., and Finke, J. H. 2002. Mechanisms of immune evasion by renal cell carcinoma: tumor-induced T-lymphocyte apoptosis and NFkappaB suppression. Urology 59:9-14.
87. Campbell, J. D., Cook, G., Robertson, S. E., Fraser, A., Boyd, K. S., Gracie, J. A., and Franklin, I. M. 2001. Suppression of IL-2-induced T cell proliferation and phosphorylation of STAT3 and STATS by tumor-derived TGF beta is reversed by IL-15. J Immunol 167:553-561.
88. Beck, C., Schreiber, H., and Rowley, D. 2001. Role of TGF-beta in immune-evasion of cancer. Microsc Res Tech 52:387-395.
89. Almand, B., Clark, J. I., Nikitina, E., van Beynen, J., English, N. R., Knight, S. C., Carbone, D. P., and Gabrilovich, D. I. 2001. Increased production of immature myeloid cells in cancer patients: a mechanism of immunosuppression in cancer. J Immunol 166:678-689.
90. Dix, A. R., Brooks, W. H., Roszman, T. L., and Morford, L. A. 1999. Immune defects observed in patients with primary malignant brain tumors. J Neuroimmunol 100:216-232.
91. Kiessling, R., Wasserman, K., Horiguchi, S., Kono, K., Sjoberg, J., Pisa, P., and Petersson, M. 1999. Tumor-induced immune dysfunction. Cancer Immunol Immunother 48:353-362.
92. Kim, H. J., Park, J. K., and Kim, Y. G. 1999. Suppression of NF-kappaB activation in normal T cells by supernatant fluid from human renal cell carcinomas. J Korean Med Sci 14:299-303.
93. Ungefroren, H., Voss, M., Bernstorff, W. V., Schmid, A., Kremer, B., and Kalthoff, H. 1999. Immunological escape mechanisms in pancreatic carcinoma. Ann N Y Acad Sci 880:243-251.
94. Fischer, J. R., Schindel, M., Bulzebruck, H., Lahm, H., Krammer, P. H., and Drings, P. 1997. Decrease of interleukin-2 secretion is a new independent prognostic factor associated with poor survival in patients with small-cell lung cancer. Ann Oncol 8:457-461.
95. Ishigami, S., Natsugoe, S., Tokuda, K., Nakajo, A., Higashi, H., Iwashige, H., Aridome, K., Hokita, S., and Aikou, T. 2002. CD3-zetachain expression of intratumoral lymphocytes is closely related to survival in gastric carcinoma patients. Cancer 94:1437-1442.
96. Marana, H. R., Silva, J. S., Andrade, J. M., and Bighetti, S. 2000. Reduced immunologic cell performance as a prognostic parameter for advanced cervical cancer. Int J Gynecol Cancer 10:67-73.
97. Gastman, B. R., Johnson, D. E., Whiteside, T. L., and Rabinowich, H. 2000. Tumor-induced apoptosis of T lymphocytes: elucidation of intracellular apoptotic events. Blood 95:2015-2023.
98. Takahashi, A., Kono, K., Amemiya, H., Iizuka, H., Fujii, H., and Matsumoto, Y. 2001. Elevated caspase-3 activity in peripheral blood T cells coexists with increased degree of T-cell apoptosis and down-regulation of TCR zeta molecules in patients with gastric cancer. Clin Cancer Res 7:74-80.
99. Mizoguchi, H., O'Shea, J. J., Longo, D. L., Loeffler, C. M., McVicar, D. W., and Ochoa, A. C. 1992. Alterations in signal transduction molecules in T lymphocytes from tumor-bearing mice. Science 258:1795-1798.
100. Horiguchi, S., Petersson, M., Nakazawa, T., Kanda, M., Zea, A. H., Ochoa, A. C., and Kiessling, R. 1999. Primary chemically induced tumors induce profound immunosuppression concomitant with apoptosis and alterations in signal transduction in T cells and NK cells. Cancer Res 59:2950-2956.
101. Schmielau, J., and Finn, O. J. 2001. Activated granulocytes and granulocyte-derived hydrogen peroxide are the underlying mechanism of suppression oft-cell function in advanced cancer patients. Cancer Res 61:4756-4760.
102. Kim, C. W., Choi, S. H., Chung, E. J., Lee, M. J., Byun, E. K., Ryu, M. H., and Bang, Y. J. 1999. Alteration of signal-transducing molecules and phenotypical characteristics in peripheral blood lymphocytes from gastric carcinoma patients. Pathobiology 67:123-128.
103. Laytragoon-Lewin, N., Porwit-MacDonald, A., Mellstedt, H., and Lewin, F. 2000. Alteration of cellular mediated cytotoxicity, T cell receptor zeta (TcR zeta) and apoptosis related gene expression in nasopharyngeal carcinoma (NPC) patients: possible clinical relevance. Anticancer Res 20:1093-1100.
104. Taylor, D. D., Bender, D. P., Gercel-Taylor, C., Stanson, J., and Whiteside, T. L. 2001. Modulation of TcR/CD3-zeta chain expression by a circulating factor derived from ovarian cancer patients. Br J Cancer 84:1624-1629.
105. Chen, X., Woiciechowsky, A., Raffegerst, S., Schendel, D., Kolb, H. J., and Roskrow, M. 2000. Impaired expression of the CD3-zeta chain in peripheral blood T cells of patients with chronic myeloid leukaemia results in an increased susceptibility to apoptosis. Br J Haematol 111:817-825.
106. Healy, C. G., Simons, J. W., Carducci, M. A., DeWeese, T. L., Bartkowski, M., Tong, K. P., and Bolton, W. E. 1998. Impaired expression and function of signal-transducing zeta chains in peripheral T cells and natural killer cells in patients with prostate cancer. Cytometry 32:109-119.
107. Valkovic, T., Dobrila, F., Melato, M., Sasso, F., Rizzardi, C., and Jonjic, N. 2002. Correlation between vascular endothelial growth factor, angiogenesis, and tumor-associated macrophages in invasive ductal breast carcinoma. Virchows Arch 440:583-588.
108. Makitie, T., Summanen, P., Tarkkanen, A., and Kivela, T. 2001. Tumor-infiltrating macrophages (CD68(+) cells) and prognosis in malignant uveal melanoma. Invest Ophthalmol Vis Sci 42:1414-1421.
109. Leek, R. D., Lewis, C. E., Whitehouse, R., Greenall, M., Clarke, J., and Harris, A. L. 1996. Association of macrophage infiltration with angiogenesis and prognosis in invasive breast carcinoma. Cancer Res 56:4625-4629.
110. Lewis, J. S., Landers, R. J., Underwood, J. C., Harris, A. L., and Lewis, C. E. 2000. Expression of vascular endothelial growth factor by macrophages is up-regulated in poorly vascularized areas of breast carcinomas. J Pathol 192:150-158.
111. Nowicki, A., Szenajch, J., Ostrowska, G., Wojtowicz, A., Wojtowicz, K., Kruszewski, A. A., Maruszynski, M., Aukerman, S. L., and Wiktor-Jedrzejczak, W. 1996. Impaired tumor growth in colony-stimulating factor 1 (CSF-1)-deficient, macrophage-deficient op/op mouse: evidence for a role of CSF-1-dependent macrophages in formation of tumor stroma. Int J Cancer 65:112-119.
112. Kamate, C., Baloul, S., Grootenboer, S., Pessis, E., Chevrot, A., Tulliez, M., Marchiol, C., Viguier, M., and Fradelizi, D. 2002. Inflammation and cancer, the mastocytoma P815 tumor model revisited: triggering of macrophage activation in vivo with pro-tumorigenic consequences. Int J Cancer 100:571-579.
113. Young, M. R., Endicott, R. A., Duffle, G. P., and Wepsic, H. T. 1987. Suppressor alveolar macrophages in mice bearing metastatic Lewis lung carcinoma tumors. J Leukoc Biol 42:682-688.
114. Billingsley, K. G., Fraker, D. L., Strassmann, G., Loeser, C., Fliot, H. M., and Alexander, H. R. 1996. Macrophage-derived tumor necrosis factor and tumor-derived of leukemia inhibitory factor and interleukin-6: possible cellular mechanisms of cancer cachexia. Ann Surg Oncol 3:29-35.
115. Bonta, I. L., and Ben-Efraim, S. 1993. Involvement of inflammatory mediators in macrophage antitumor activity. J Leukoc Biol 54:613-626.
116. Bhaumik, S., and Khar, A. 1998. Induction of nitric oxide production by the peritoneal macrophages after intraperitoneal or subcutaneous transplantation of AK-5 tumor. Nitric Oxide 2:467-474.
117. Lewis, J. G., and Adams, D. O. 1987. Inflammation, oxidative DNA damage, and carcinogenesis. Environ Health Perspect 76:19-27.
118. Kono, K., Salazar-Onfray, F., Petersson, M., Hansson, J., Masucci, G., Wasserman, K., Nakazawa, T., Anderson, P., and Kiessling, R. 1996. Hydrogen peroxide secreted by tumor-derived macrophages down-modulates signal-transducing zeta molecules and inhibits tumor-specific T cell- and natural killer cell-mediated cytotoxicity. Eur J Immunol 26:1308-1313.
119. Murr, C., Widner, B., Wirleitner, B., and Fuchs, D. 2002. Neopterin as a marker for immune system activation. Curr Drug Metab 3:175-187.
120. Whisler, R. L., Gray, L. S., and Hackshaw, K. V. 2002. Rheumatology, a clinical overview. Clin Podiatr Med Surg 19:149-161, vii.
121. Ishihara, K., and Hirano, T. 2002. IL-6 in autoimmune disease and chronic inflammatory proliferative disease. Cytokine Growth Factor Rev 13:357.
122. Mahmoud, F. A., and Rivera, N. I. 2002. The role of C-reactive protein as a prognostic indicator in advanced cancer. Curr Oncol Rep 4:250-255.
123. Smith, P. C., Hobisch, A., Lin, D. L., Culig, Z., and Keller, E. T. 2001. Interleukin-6 and prostate cancer progression. Cytokine Growth Factor Rev 12:33-40.
124. Rutkowski, P., Kaminska, J., Kowalska, M., Ruka, W., and Steffen, J. 2002. Cytokine serum levels in soft tissue sarcoma patients: correlations with clinico-pathological features and prognosis. Int J Cancer 100:463-471.
125. Kallio, J. P., Tammela, T. L., Marttinen, A. T., and Kellokumpu-Lehtinen, P. L. 2001. Soluble immunological parameters and early prognosis of renal cell cancer patients. J Exp Clin Cancer Res 20:523-528.
126. Ljungberg, B., Grankvist, K., and Rasmuson, T. 1997. Serum interleukin-6 in relation to acute-phase reactants and survival in patients with renal cell carcinoma. Eur J Cancer 33:1794-1798.
127. Fearon, K. C., Barber, M. D., Falconer, J. S., McMillan, D. C., Ross, J. A., and Preston, T. 1999. Pancreatic cancer as a model: inflammatory mediators, acute-phase response, and cancer cachexia. World J Surg 23:584-588.
128. McMillan, D. C., Watson, W. S., O'Gorman, P., Preston, T., Scott, H. R., and McArdle, C. S. 2001. Albumin concentrations are primarily determined by the body cell mass and the systemic inflammatory response in cancer patients with weight loss. Nutr Cancer 39:210-213.
129. Oya, M., Akiyama, Y., Okuyama, T., and Ishikawa, H. 2001. High preoperative plasma D-dimer level is associated with advanced tumor stage and short survival after curative resection in patients with colorectal cancer. Jpn J Clin Oncol 31:388-394.
130. Ferrigno, D., Buccheri, G., and Ricca, I. 2001. Prognostic significance of blood coagulation tests in lung cancer. Eur Respir J 17:667-673.
131. Blackwell, K., Haroon, Z., Broadwater, G., Berry, D., Harris, L., Iglehart, J. D., Dewhirst, M., and Greenberg, C. 2000. Plasma D-dimer levels in operable breast cancer patients correlate with clinical stage and axillary lymph node status. J Clin Oncol 18:600-608.
132. Tabuchi, T., Ubukata, H., Saniabadi, A. R., and Soma, T. 1999. Granulocyte apheresis as a possible new approach in cancer therapy: A pilot study involving two cases. Cancer Detect Prev 23:417-421.
133. Oberholzer, A., Oberholzer, C., and Moldawer, L. L. 2001. Sepsis syndromes: understanding the role of innate and acquired immunity. Shock 16:83-96.
134. Heidecke, C. D., Hensler, T., Weighardt, H., Zantl, N., Wagner, H., Siewert, J. R., and Holzmann, B. 1999. Selective defects of T lymphocyte function in patients with lethal intraabdominal infection. Am J Surg 178:288-292.
135. Elgert, K. D., Alleva, D. G., and Mullins, D. W. 1998. Tumor-induced immune dysfunction: the macrophage connection. J Leukoc Biol 64:275-290.
136. De la Fuente, M., and Victor, V. M. 2001. Ascorbic acid and N-acetylcysteine improve in vitro the function of lymphocytes from mice with endotoxin-induced oxidative stress. Free Radic Res 35:73-84.
137. Malmberg, K. J., Lenkei, R., Petersson, M., Ohlum, T., Ichihara, F., Glimelius, B., Frodin, J. E., Masucci, G., and Kiessling, R. 2002. A short-term dietary supplementation of high doses of vitamin E increases T helper 1 cytokine production in patients with advanced colorectal cancer. Clin Cancer Res 8:1772-1778.
138. Heller, A. R., Groth, G., Heller, S. C., Breitkreutz, R., Nebe, T., Quintel, M., and Koch, T. 2001. N-acetylcysteine reduces respiratory burst but augments neutrophil phagocytosis in intensive care unit patients. Crit Care Med 29:272-276.
139. Van Schooten, F. J., Nia, A. B., De Flora, S., D'Agostini, F., Izzotti, A., Camoirano, A., Balm, A. J., Dallinga, J. W., Bast, A., Haenen, G. R., et al. 2002. Effects of oral administration of N-acetyl-L-cysteine: a multi-biomarker study in smokers. Cancer Epidemiol Biomarkers Prev 11:167-175.
140. Albini, A., Morini, M., D'Agostini, F., Ferrari, N., Campelli, F., Arena, G., Noonan, D. M., Pesce, C., and De Flora, S. 2001. Inhibition of angiogenesis-driven Kaposi's sarcoma tumor growth in nude mice by oral N-acetylcysteine. Cancer Res 61:8171-8178.
141. Leibovitz, B., and Siegel, B. V. 1978. Ascorbic acid, neutrophil function, and the immune response. Int J Vitam Nutr Res 48:159-164.
142. Siegel, B. V. 1974. Enhanced interferon response to murine leukemia virus by ascorbic acid. Infect Immun 10:409-410.
143. Riordan, H. D., Hunninghake, R. B., Riordan, N. H., Jackson, J. J., Meng, X., Taylor, P., Casciari, J. J., Gonzalez, M. J., Miranda-Massari, J. R., Mora, E. M., et al. 2003. Intravenous ascorbic acid: protocol for its application and use. P R Health Sci J 22:287-290.
144. Folkers, K., and Wolaniuk, A. 1985. Research on coenzyme Q10 in clinical medicine and in immunomodulation. Drugs Exp Clin Res 11:539-545.
145. Ravindranath, M. H., Muthugounder, S., Presser, N., and Viswanathan, S. 2004. Anticancer therapeutic potential of soy isoflavone, genistein. Adv Exp Med Biol 546:121-165.
146. Li, T., Sheng, L., Fan, G. X., and Yuan, Y. K. 2005. [Preliminary study on anti-tumor function of resveratrol and its immunological mechanism.]. Xi Bao Yu Fen Zi Mian Yi Xue Za Zhi 21:575-579.
147. Lan, R. Y., Ansari, A. A., Lian, Z. X., and Gershwin, M. E. 2005. Regulatory T cells: development, function and role in autoimmunity. Autoimmun Rev 4:351-363.
148. Anderson, M. S., Venanzi, E. S., Chen, Z., Berzins, S. P., Benoist, C., and Mathis, D. 2005. The cellular mechanism of Aire control of T cell tolerance. Immunity 23:227-239.
149. Watanabe, N., Wang, Y. H., Lee, H. K., Ito, T., Cao, W., and Liu, Y. J. 2005. Hassall's corpuscles instruct dendritic cells to induce CD4+CD25+ regulatory T cells in human thymus. Nature 436:1181-1185.
150. Min, W. P., Zhou, D., Ichim, T. E., Strejan, G. H., Xia, X., Yang, J., Huang, X., Garcia, B., White, D., Dutartre, P., et al. 2003. Inhibitory feedback loop between tolerogenic dendritic cells and regulatory T cells in transplant tolerance. J Immunol 170:1304-1312.
151. Stassen, M., Schmitt, E., and Jonuleit, H. 2004. Human CD(4+)CD(25+) regulatory T cells and infectious tolerance. Transplantation 77:S23-25.
152. Green, E. A., Gorelik, L., McGregor, C. M., Tran, E. H., and Flavell, R. A. 2003. CD4+CD25+ T regulatory cells control anti-islet CD8+ T cells through TGF-beta-TGF-beta receptor interactions in type 1 diabetes. Proc Natl Acad Sci USA 100:10878-10883.
153. Levings, M. K., Sangregorio, R., and Roncarolo, M. G. 2001. Human cd25(+)cd4(+) t regulatory cells suppress naive and memory T cell proliferation and can be expanded in vitro without loss of function. J Exp Med 193:1295-1302.
154. Azuma, T., Takahashi, T., Kunisato, A., Kitamura, T., and Hirai, H. 2003. Human CD4+ CD25+ regulatory T cells suppress NKT cell functions. Cancer Res 63:4516-4520.
155. Serra, P., Amrani, A., Yamanouchi, J., Han, B., Thiessen, S., Utsugi, T., Verdaguer, J., and Santamaria, P. 2003. CD40 ligation releases immature dendritic cells from the control of regulatory CD4+CD25+ T cells. Immunity 19:877-889.
156. Huber, S., and Schramm, C. 2006. TGF-beta and CD4+CD25+ regulatory T cells. Front Biosci 11:1014-1023.
157. Gondek, D. C., Lu, L. F., Quezada, S. A., Sakaguchi, S., and Noelle, R. J. 2005. Cutting edge: contact-mediated suppression by CD4+CD25+ regulatory cells involves a granzyme B-dependent, perforin-independent mechanism. J Immunol 174:1783-1786.
158. Mekala, D. J., Alli, R. S., and Geiger, T. L. 2005. IL-10-dependent infectious tolerance after the treatment of experimental allergic encephalomyelitis with redirected CD4+CD25+ T lymphocytes. Proc Natl Acad Sci USA 102:11817-11822.
159. Morgan, M. E., Sutmuller, R. P., Witteveen, H. J., van Duivenvoorde, L. M., Zanelli, E., Melief, C. J., Snijders, A., Offringa, R., de Vries, R. R., and Toes, R. E. 2003. CD25+ cell depletion hastens the onset of severe disease in collagen-induced arthritis. Arthritis Rheum 48:1452-1460.
160. Nagai, H., Horikawa, T., Hara, I., Fukunaga, A., Oniki, S., Oka, M., Nishigori, C., and Ichihashi, M. 2004. In vivo elimination of CD25+ regulatory T cells leads to tumor rejection of B16F10 melanoma, when combined with interleukin-12 gene transfer. Exp Dermatol 13:613-620.
161. Dannull, J., Su, Z., Rizzieri, D., Yang, B. K., Coleman, D., Yancey, D., Zhang, A., Dahm, P., Chao, N., Gilboa, E., et al. 2005. Enhancement of vaccine-mediated antitumor immunity in cancer patients after depletion of regulatory T cells. J Clin Invest.
162. Banuelos, S. J., Markees, T. G., Phillips, N. E., Appel, M. C., Cuthbert, A., Leif, J., Mordes, J. P., Shultz, L. D., Rossini, A. A., and Greiner, D. L. 2004. Regulation of skin and islet allograft survival in mice treated with costimulation blockade is mediated by different CD4+ cell subsets and different mechanisms. Transplantation 78:660-667.
163. Taylor, P. A., Noelle, R. J., and Blazar, B. R. 2001. CD4(+)CD25(+) immune regulatory cells are required for induction of tolerance to alloantigen via costimulatory blockade. J Exp Med 193:1311-1318.
164. Dubois, B., Chapat, L., Goubier, A., Papiernik, M., Nicolas, J. F., and Kaiserlian, D. 2003. Innate CD4+CD25+ regulatory T cells are required for oral tolerance and inhibition of CD8+ T cells mediating skin inflammation. Blood 102:3295-3301.
165. Yamashiro, H., Hozumi, N., and Nakano, N. 2002. Development of CD25(+) T cells secreting transforming growth factor-beta1 by altered peptide ligands expressed as self-antigens. Int Immunol 14:857-865.
166. Ehrenstein, M. R., Evans, J. G., Singh, A., Moore, S., Warnes, G., Isenberg, D. A., and Mauri, C. 2004. Compromised function of regulatory T cells in rheumatoid arthritis and reversal by anti-TNFalpha therapy. J Exp Med 200:277-285.
167. Toubi, E., Kessel, A., Mahmudov, Z., Hallas, K., Rozenbaum, M., and Rosner, I. 2005. Increased Spontaneous Apoptosis of CD4+CD25+ T Cells in Patients with Active Rheumatoid Arthritis Is Reduced by Infliximab. Ann N Y Acad Sci 1051:506-514.
168. Zorn, E., Kim, H. T., Lee, S. J., Floyd, B. H., Litsa, D., Arumugarajah, S., Bellucci, R., Alyea, E. P., Antin, J. H., Soiffer, R. J., et al. 2005. Reduced frequency of FOXP3+ CD4+CD25+ regulatory T cells in patients with chronic graft-versus-host disease. Blood 106:2903-2911.
169. Haas, J., Hug, A., Viehover, A., Fritzsching, B., Falk, C. S., Filser, A., Vetter, T., Milkova, L., Korporal, M., Fritz, B., et al. 2005. Reduced suppressive effect of CD4(+)CD25(high) regulatory T cells on the T cell immune response against myelin oligodendrocyte glycoprotein in patients with multiple sclerosis. Eur J Immunol 35:3343-3352.
170. Huan, J., Culbertson, N., Spencer, L., Bartholomew, R., Burrows, G. G., Chou, Y. K., Bourdette, D., Ziegler, S. F., Offner, H., and Vandenbark, A. A. 2005. Decreased FOXP3 levels in multiple sclerosis patients. J Neurosci Res 81:45-52.
171. Viglietta, V., Baecher-Allan, C., Weiner, H. L., and Hafler, D. A. 2004. Loss of functional suppression by CD4+CD25+ regulatory T cells in patients with multiple sclerosis. J Exp Med 199:971-979.
172. Hong, J., Li, N., Zhang, X., Zheng, B., and Zhang, J. Z. 2005. Induction of CD4+CD25+ regulatory T cells by copolymer-I through activation of transcription factor Foxp3. Proc Natl Acad Sci USA 102:6449-6454.
173. Vandenbark, A. A. 2005. TCR peptide vaccination in multiple sclerosis: boosting a deficient natural regulatory network that may involve TCR-specific CD4+CD25+ Treg cells. Curr Drug Targets Inflamm Allergy 4:217-229.
174. Sanchez-Ramon, S., Navarro, A. J., Aristimuno, C., Rodriguez-Mahou, M., Bellon, J. M., Fernandez-Cruz, E., and de Andres, C. 2005. Pregnancy-induced expansion of regulatory T-lymphocytes may mediate protection to multiple sclerosis activity. Immunol Lett 96:195-201.
175. Fu, T., Shen, Y., and Fujimoto, S. 2000. Tumor-specific CD4(+) suppressor T-cell clone capable of inhibiting rejection of syngeneic sarcoma in A/J mice. Int J Cancer 87:680-687.
176. Antony, P. A., Piccirillo, C. A., Akpinarli, A., Finkelstein, S. E., Speiss, P. J., Surman, D. R., Palmer, D. C., Chan, C. C., Klebanoff, C. A., Overwijk, W. W., et al. 2005. CD8+ T cell immunity against a tumor/self-antigen is augmented by CD4+T helper cells and hindered by naturally occurring T regulatory cells. J Immunol 174:2591-2601.
177. Nicholl, M., Lodge, A., Brown, I., Sugg, S. L., and Shilyansky, J. 2004. Restored immune response to an MHC-II-Restricted antigen in tumor-bearing hosts after elimination of regulatory T cells. J Pediatr Surg 39:941-946; discussion 941-946.
178. Sasada, T., Kimura, M., Yoshida, Y., Kanai, M., and Takabayashi, A. 2003. CD4+CD25+ regulatory T cells in patients with gastrointestinal malignancies: possible involvement of regulatory T cells in disease progression. Cancer 98:1089-1099.
179. Unitt, E., Rushbrook, S. M., Marshall, A., Davies, S., Gibbs, P., Morris, L. S., Coleman, N., and Alexander, G. J. 2005. Compromised lymphocytes infiltrate hepatocellular carcinoma: the role of T-regulatory cells. Hepatology 41:722-730.
180. Jonuleit, H., and Schmitt, E. 2005. Regulatory T-cells in antitumor therapy: isolation and functional testing of CD4+CD25+ regulatory T-cells. Methods Mol Med 109:285-296.
181. Pasare, C., and Medzhitov, R. 2003. Toll pathway-dependent blockade of CD4+CD25+ T cell-mediated suppression by dendritic cells. Science 299:1033-1036.
182. Peng, G., Guo, Z., Kiniwa, Y., Voo, K. S., Peng, W., Fu, T., Wang, D. Y., Li, Y., Wang, H. Y., and Wang, R. F. 2005. Toll-like receptor 8-mediated reversal of CD4+ regulatory T cell function. Science 309:1380-1384.
183. Shackleton, M., Davis, I. D., Hopkins, W., Jackson, H., Dimopoulos, N., Tai, T., Chen, Q., Parente, P., Jefford, M., Masterman, K. A., et al. 2004. The impact of imiquimod, a Toll-like receptor-7 ligand (TLR7L), on the immunogenicity of melanoma peptide vaccination with adjuvant Flt3 ligand. Cancer Immun 4:9.
184. Nair, S., McLaughlin, C., Weizer, A., Su, Z., Boczkowski, D., Dannull, J., Vieweg, J., and Gilboa, E. 2003. Injection of immature dendritic cells into adjuvant-treated skin obviates the need for ex vivo maturation. J Immunol 171:6275-6282.
185. Li, M., Qian, H., Ichim, T. E., Ge, W. W., Popov, I. A., Rycerz, K., Neu, J., White, D., Zhong, R., and Min, W. P. 2004. Induction of RNA interference in dendritic cells. Immunol Res 30:215-230.
186. Mellor, A. 2005. Indoleamine 2,3 dioxygenase and regulation of T cell immunity. Biochem Biophys Res Commun 338:20-24.
187. Kwidzinski, E., Bunse, J., Aktas, O., Richter, D., Mutlu, L., Zipp, F., Nitsch, R., and Bechmann, I. 2005. Indolamine 2,3-dioxygenase is expressed in the CNS and down-regulates autoimmune inflammation. Faseb J 19:1347-1349.
188. Yang, A. S., and Lattime, E. C. 2003. Tumor-induced interleukin 10 suppresses the ability of splenic dendritic cells to stimulate CD4 and CD8 T-cell responses. Cancer Res 63:2150-2157.
189. Chen, W., and Wahl, S. M. 2003. TGF-beta: the missing link in CD4+CD25+ regulatory T cell-mediated immunosuppression. Cytokine Growth Factor Rev 14:85-89.
190. Ryan, A. E., Shanahan, F., O'Connell, J., and Houston, A. M. 2005. Addressing the “Fas counterattack” controversy: blocking fas ligand expression suppresses tumor immune evasion of colon cancer in vivo. Cancer Res 65:9817-9823.
191. Ohm, J. E., Gabrilovich, D. I., Sempowski, G. D., Kisseleva, E., Parman, K. S., Nadaf, S., and Carbone, D. P. 2003. VEGF inhibits T-cell development and may contribute to tumor-induced immune suppression. Blood 101:4878-4886.
192. Paulukat, J., Bosmann, M., Nold, M., Garkisch, S., Kampfer, H., Frank, S., Raedle, J., Zeuzem, S., Pfeilschifter, J., and Muhl, H. 2001. Expression and release of IL-18 binding protein in response to IFN-gamma. J Immunol 167:7038-7043.
193. Chan, A. K., Lockhart, D. C., von Bernstorff, W., Spanjaard, R. A., Joo, H. G., Eberlein, T. J., and Goedegebuure, P. S. 1999. Soluble MUC1 secreted by human epithelial cancer cells mediates immune suppression by blocking T-cell activation. Int J Cancer 82:721-726.
194. Hsu, T. L., Wu, Y. Y., Chang, Y. C., Yang, C. Y., Lai, M. Z., Su, W. B., and Hsieh, S. L. 2005. Attenuation of Th1 response in decoy receptor 3 transgenic mice. J Immunol 175:5135-5145.
195. Zhu, L. X., Sharma, S., Gardner, B., Escuadro, B., Atianzar, K., Tashkin, D. P., and Dubinett, S. M. 2003. IL-10 mediates sigma 1 receptor-dependent suppression of antitumor immunity. J Immunol 170:3585-3591.
196. Gray, C. P., Arosio, P., and Hersey, P. 2003. Association of increased levels of heavy-chain ferritin with increased CD4+ CD25+ regulatory T-cell levels in patients with melanoma. Clin Cancer Res 9:2551-2559.
197. Smith, G. R., and Missailidis, S. 2004. Cancer, inflammation and the AT1 and AT2 receptors. J Inflamm (Loud) 1:3.
198. Ostrand-Rosenberg, S., Sinha, P., Danna, E. A., Miller, S., Davis, C., and Dissanayake, S. K. 2004. Antagonists of tumor-specific immunity: tumor-induced immune suppression and host genes that co-opt the anti-tumor immune response. Breast Dis 20:127-135.
199. Xu, C., Jung, M., Burkhardt, M., Stephan, C., Schnorr, D., Loening, S., Jung, K., Dietel, M., and Kristiansen, G. 2005. Increased CD59 protein expression predicts a PSA relapse in patients after radical prostatectomy. Prostate 62:224-232.
200. Hill, J. A., Ichim, T. E., Kusznieruk, K. P., Li, M., Huang, X., Yan, X., Zhong, R., Cairns, E., Bell, D. A., and Min, W. P. 2003. Immune modulation by silencing IL-12 production in dendritic cells using small interfering RNA. J Immunol 171:691-696.
201. Ichim, T. E., Li, M., Qian, H., Popov, I. A., Rycerz, K., Zheng, X., White, D., Zhong, R., and Min, W. P. 2004. RNA interference: a potent tool for gene-specific therapeutics. Am J Transplant 4:1227-1236.
202. Klein, C., Bock, C. T., Wedemeyer, H., Wustefeld, T., Locarnini, S., Dienes, H. P., Kubicka, S., Manns, M. P., and Trautwein, C. 2003. Inhibition of hepatitis B virus replication in vivo by nucleoside analogues and siRNA. Gastroenterology 125:9-18.
203. Ichim, T. E., Zhong, R., and Min, W. P. 2003. Prevention of allograft rejection by in vitro generated tolerogenic dendritic cells. Transpl Immunol 11:295-306.
204. Bhattacharya, S., Dhillon, A. P., Winslet, M. C., Davidson, B. R., Shukla, N., Gupta, S. D., Al-Mufti, R., and Hobbs, K. E. 1996. Human liver cancer cells and endothelial cells incorporate iodised oil. Br J Cancer 73:877-881.
205. Vogl, T. J., Wetter, A., Lindemayr, S., and Zangos, S. 2005. Treatment of unresectable lung metastases with transpulmonary chemoembolization: preliminary experience. Radiology 234:917-922.
206. Di Stefano, D. R., de Baere, T., Denys, A., Hakime, A., Gorin, G., Gillet, M., Saric, J., Trillaud, H., Petit, P., Bartoli, J. M., et al. 2005. Preoperative percutaneous portal vein embolization: evaluation of adverse events in 188 patients. Radiology 234:625-630.

1. A method of treating cancer in a cancer patient in need thereof comprising:
Admixing a concentration of a gene silencing agent with a clinically applicable localizing agent and a single or plurality of agents capable of causing localized cell death;
Administering said combination directly into the tumor and/or arteries providing the tumor with blood supply; and
Administering an embolizing agent in the proximity of the tumor and/or directly into the arteries providing the tumor with blood supply.

2. The method of claim 1 wherein the gene silencing agent is selected from a group consisting of: a) siRNA, b) ddRNA, and c) shRNA.

3. The method of claim 1 wherein said agent capable of causing cell death is a chemotherapeutic or radiotherapeutic agent.

4. The method of claim 1 wherein the localizing agent is an iodinated oil mixture

5. The method of claim 1 wherein the localizing agent is lipiodol.

6. The method of claim 1 wherein the embolizing agent is selected from a group consisting of: Avitene, Gelfoam, Occlusin and Angiostat.

7. The method of claim 2 wherein said siRNA is administered in a form selected from the group consisting of: DNA plasmids capable of transcribing hairpin loop RNA which is subsequently cleaved by endogenous cellular processes into short interfering RNA, double stranded RNA chemically synthesized oligonucleotides, and in vitro generated siRNA fragments from mRNA.

8. The method of claim 7 wherein the short interfering RNA is targeted to one or more mRNA selected from the group consisting of: IDO, IL-4, IL-10, TGF-β, FGF, NR2F6, and VEGF.

9. The method of claim 7 wherein the short interfering RNA is targeted to one or more mRNA selected from the following group: a) brother of the regulatory of imprinted sites (BORIS); b) NR2F6; c) NR2F2

10. The method of claim 2, wherein said siRNA is comprised of one strand possessing the sequence GGAAAUACCA CGAUGCAAAT (SEQ ID NO: 1).

11. The method of claim 2, wherein said siRNA is comprised of one strand possessing the sequence GGCAAGUAAA UUGAAGCGCT (SEQ ID NO: 2).

12. A pharmaceutical composition capable of delivering nucleic acids capable of gene silencing in tumors comprising of:
a nucleic acid
a clinically applicable localizing agent;
an agent capable of causing cell death; and
an embolizing agent

13. The pharmaceutical composition of claim 12 wherein said gene silencing agent is selected from a group consisting of: a) siRNA; b) ddRNA; c) shRNA.

14. The pharmaceutical composition of claim 13 wherein said agent capable of causing cell death is a chemotherapeutic or radiotherapeutic agent.

15. The pharmaceutical composition of claim 12 wherein the localizing agent is an iodinated oil mixture

16. The pharmaceutical composition of claim 15 wherein the localizing agent is lipiodol.

17. The pharmaceutical composition of claim 12 wherein the embolizing agent is selected from a group consisting of: Avitene, Gelfoam, Occlusin and Angiostat.

18. The pharmaceutical composition of claim 13 wherein said siRNA is administered in the a form selected from the group consisting of: DNA plasmids capable of transcribing hairpin loop RNA which is subsequently cleaved by endogenous cellular processes into short interfering RNA, double stranded RNA chemically synthesized oligonucleotides, and in vitro generated siRNA fragments from mRNA.

19. The pharmaceutical composition of claim 13 wherein the short interfering RNA is targeted to one or more mRNA selected from the groups consisting of: IDO, IL-4, IL-10, TGF-β, FGF, and VEGF.


SEQUENCE LISTING (2 sequences)

SEQ ID NO: 1 - DNA, 20 bp, Artificial Sequence
  Description of Artificial Sequence Synthetic oligonucleotide
ggaaauaccacgaugcaaat

SEQ ID NO: 2 - DNA, 20 bp, Artificial Sequence
  Description of Artificial Sequence Synthetic oligonucleotide
ggcaaguaaauugaagcgct